(19) United States
(12) Patent Application Publication
(10) Pub. No.: US 2008/0306308 A1
(43) Pub. Date: Dec. 11, 2008

(54) PROCESS FOR PREPARING FLUORINE-CONTAINING ALKOXYALKANE

(76) Inventors: Tetsuo Nanno, Osaka (JP); Masaki Deguchi, Hyogo (JP)

Correspondence Address: MCDERMOTT WILL & EMERY LLP, 600 13TH STREET, NW, WASHINGTON, DC, 20005-3096, US

(21) Appl. No.: 12/133,050

(22) Filed: Jun. 4, 2008

Related U.S. Application Data
(60) Provisional application No. 60/943,408, filed on Jun. 12, 2007.

(30) Foreign Application Priority Data
Jun. 6, 2007 (JP) 2007-150525

(51) Int. Cl. C07C 43/04

(52) U.S. Cl. 568/615

(57) ABSTRACT

A process for preparing a fluorine-containing alkoxyalkane represented by the general formula (1) R¹—O—R²—O—R³ where at least one of R¹, R² and R³ contains one or more fluorine atoms. An alcohol with the highest acidity selected from the group consisting of the compounds represented by the general formula (2) R¹—OH, the general formula (3) R³—O—R²—OH, the general formula (4) R¹—O—R²—OH, and the general formula (5) R³—OH is reacted with at least one selected from the group consisting of the compounds represented by the general formula (6) Lg-R²—O—R³, the general formula (7) Lg-R¹, the general formula (8) Lg-R³, and the general formula (9) Lg-R²—O—R¹ where Lg represents an anionic leaving group in the presence of a basic compound.

### FIELD OF THE INVENTION

The invention relates to a process for preparing a fluorine-containing alkoxyalkane, and more particularly, to a process for preparing a fluorine-containing alkoxyalkane containing a plurality of ether groups.

### BACKGROUND OF THE INVENTION

With the reduction in the size of electronic devices, batteries used as the main power source or back-up power source therefor are required to have high energy density. With respect to such requirement, non-aqueous electrolyte secondary batteries have recently been receiving attention. Non-aqueous electrolyte secondary batteries have higher voltage and higher energy density than conventional secondary batteries including aqueous electrolyte. Thus, non-aqueous electrolyte secondary batteries are also expected to be used as the power source for hybrid electric vehicles, and required to provide higher power, longer life, and high reliability.

A non-aqueous electrolyte secondary battery includes a positive electrode, a negative electrode, a separator interposed between the positive electrode and the negative electrode, and a non-aqueous electrolyte. The non-aqueous electrolyte includes a non-aqueous solvent and a solute dissolved therein. To provide the non-aqueous electrolyte secondary battery with higher power and longer life, it is effective to reduce the viscosity of the non-aqueous electrolyte.

Patent Document 1 proposes non-aqueous solvents which include compounds prepared by fluorinating dialkoxyethanes represented by the general formula:

ROCH₂CH₂OR′

where R is a univalent group represented by CF₃CH₂— or (CF₃)₂CH—, and R′ is —CH₃, —C₂H₅, —CH(CH₃)₂, —CH₂CF₃ or (CF₃)₂CH—. Since a non-aqueous solvent comprising a fluorinated dialkoxyethane has a low viscosity, it has the effect of increasing power and oxidation resistance.

Among them, CH₃OCH₂CH₂OCH₂CF₃, which has a low viscosity (0.748 c.p.) and a high relative dielectric constant (15.7 (5 kHz)), is considered preferable as a non-aqueous solvent.

In the preparation process of Patent Document 1, ethylene glycol monoether is reacted with a p-toluenesulfonate of a fluorinated alcohol in the presence of dimethyl sulfoxide (DMSO) and NaOH. This reaction is expressed by the formula [1].

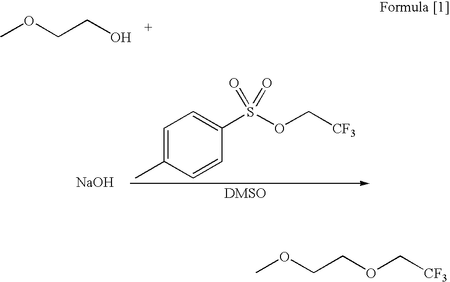

Non-Patent Document 1 proposes the use of CH₃CH₂OCH₂CH₂OCH₂CF₃ and CF₃CH₂OCH₂CH₂OCH₂CF₃ in addition to CH₃OCH₂CH₂OCH₂CF₃ as non-aqueous solvents. Also, it reports that the inclusion of fluorine in such a non-aqueous solvent allows efficient reductive deposition and oxidative dissolution of lithium.

Non-Patent Documents 2 and 3 describe the physical properties of CH₃OCH₂CH₂OCH₂CHF₂, CH₃OCH₂CH₂OCH₂CF₃, and CH₃CH₂OCH₂CH₂OCH₂CF₃.

Non-Patent Documents 4 and 5 describe information on the acidity of hydroxyl groups of alcohols.

Patent Document 1: Japanese Laid-Open Patent Publication No. Hei 1-117838

Non-Patent Document 1: Abstracts of the 8^{th} Meeting of Association of Chemical Battery Material (Kagaku Denti Zairyo Kenkyukai), page 67

Non-Patent Document 2: Abstracts of the 72th Meeting of the Electrochemical Society of Japan, page 313

Non-Patent Document 3: Abstracts of the 73th Meeting of the Electrochemical Society of Japan, page 242

Non-Patent Document 4: Journal of Organic Chemistry, 1980, vol. 45, page 3295

Non-Patent Document 5: The Journal of Biological Chemistry, 1990, vol. 265, page 22101

In the preparation process of Patent Document 1, by-products such as diethyleneglycol dimethyl ether (diglyme) and bis(2,2,2-trifluoroethyl)ether are produced. Also, since water is produced by the reaction, the water needs to be removed. Thus, the yield of the desired compound is considered low. In Patent Document 1, for example, the yield in Example 1 is approximately 24%.

The formula [2] represents a side reaction in which 2,2,2-trifluoroethoxymethoxyethane, which is a desired product, reacts with 2-methoxyethoxide, which is an activated reactive species, to cause elimination of 2,2,2-trifluoroethoxide therefrom. This side reaction is thought to involve the production of diglyme and bis(2,2,2-trifluoroethyl)ether.

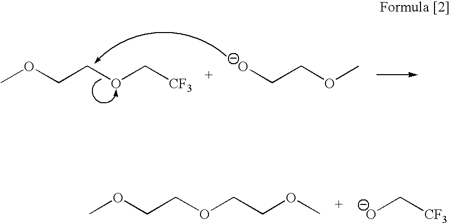

### BRIEF SUMMARY OF THE INVENTION

In view of the above-described problems, an object of the invention is to provide a process for preparing a fluorine-containing alkoxyalkane with a good yield in which the production of by-products is suppressed.

The invention relates to a process (also referred to as “preparation process A”) for preparing a fluorine-containing alkoxyalkane represented by the general formula (1):

R¹—O—R²—O—R³ (1)

where each of R¹ and R³ is a C₁ to C₆ alkyl group in which part of the hydrogen atoms may be replaced with one or more fluorine atoms, R² is a C₂ to C₄ alkylene group in which part of the hydrogen atoms may be replaced with one or more fluorine atoms, and at least one of R¹, R² and R³ contains one or more fluorine atoms. The process includes the step of reacting a first compound with a second compound in a basic compound or a solvent containing a basic compound, the second compound being reactive with the first compound. The first compound is an alcohol with the highest acidity selected from the group consisting of the compounds represented by the general formulas (2) to (5):

R¹—OH (2)

R³—O—R²—OH (3)

R¹—O—R²—OH (4)

R³—OH (5)

The second compound is one selected from the group consisting of the compounds represented by the general formulas (6) to (9):

Lg-R²—O—R³ (6)

Lg-R¹ (7)

Lg-R³ (8)

Lg-R²—O—R¹ (9)

where Lg represents an anionic leaving group. The second compound is a compound that reacts with the first compound to yield the fluorine-containing alkoxyalkane.

According to the preparation process A, since the conjugate base of the first compound does not seemingly make a nucleophilic attack on the produced fluorine-containing alkoxyalkane, the production of by-products can be suppressed. It is thus possible to achieve a high yield.

The combination of the first compound and the second compound corresponding to the first compound is a combination of compounds of the general formula (2) and the general formula (6), a combination of compounds of the general formula (3) and the general formula (7), a combination of compounds of the general formula (4) and the general formula (8), or a combination of compounds of the general formula (5) and the general formula (9).

That is, of the general formulas (2) to (5), when a compound of the general formula (2) has the highest acidity, a compound of the general formula (2) is used as the first compound, and a compound of the general formula (6) is used as the second compound.

Likewise, of the general formulas (2) to (5), when the general formula (3) has the highest acidity, a compound of the general formula (3) is used as the first compound, and a compound of the general formula (7) is used as the second compound.

Also, of the general formulas (2) to (5), when the general formula (4) has the highest acidity, a compound of the general formula (4) is used as the first compound, and a compound of the general formula (8) is used as the second compound.

Also, of the general formulas (2) to (5), when the general formula (5) has the highest acidity, a compound of the general formula (5) is used as the first compound, and a compound of the general formula (9) is used as the second compound.

Preferably, the solvent serving as the reaction field of the first compound and the second compound includes at least one selected from the group consisting of diethyl ether, tetrahydrofuran, dimethyl sulfoxide, acetonitrile, 2-methylpyrrolidinone, pyridine, picoline, lutidine, and dioxane.

In the preparation process A, it is preferable to add the first compound and the basic compound to the solvent and thereafter add the second compound to the solvent. If the second compound and the basic compound can coexist, it is also possible to add the second compound and the basic compound to the solvent and thereafter add the first compound to the solvent.

The invention also pertains to a process (also referred to as “preparation process B”) for preparing a fluorine-containing alkoxyalkane represented by the general formula (10):

R⁵—O—R⁴—O—R⁵ (10)

where R⁴ is a C₂ to C₄ alkylene group in which part of the hydrogen atoms may be replaced with one or more fluorine atoms, R⁵ is a C₁ to C₆ alkyl group in which part of the hydrogen atoms may be replaced with one or more fluorine atoms, and at least one of R⁴ and R⁵ includes one or more fluorine atoms. The process includes the step of reacting a third compound with a fourth compound in a basic compound or a solvent containing a basic compound, the fourth compound being reactive with the third compound. The third compound is an alkoxide which is the conjugate base of an alcohol with a higher acidity selected from the group consisting of the compounds represented by the general formulas (11) and (12):

HO—R⁴—OH (11)

R⁵—OH (12)

The fourth compound is one selected from the group consisting of the compounds represented by the general formulas (13) and (14):

Lg-R⁵ (13)

Lg-R⁴-Lg′ (14)

where each of Lg and Lg′ is an anionic leaving group. The fourth compound is a compound that reacts with the third compound to yield the fluorine-containing alkoxyalkane.

According to the preparation process B, since the third compound does not seemingly make a nucleophilic attack on the produced fluorine-containing alkoxyalkane, the production of by-products can be suppressed. It is thus possible to synthesize a symmetric fluorine-containing alkoxyalkane with a high yield.

The combination of the third compound and the fourth compound is a combination of an alkoxide which is the conjugate base of the general formula (11) and a compound of the general formula (13), or a combination of an alkoxide which is the conjugate base of the general formula (12) and a compound of the general formula (14).

That is, when a compound of the general formula (11) has a higher acidity than the general formula (12), an alkoxide which is the conjugate base of the general formula (11) is used as the third compound, and a compound of the general formula (13) is used as the fourth compound.

Likewise, when the general formula (12) has a higher acidity than the general formula (11), an alkoxide which is the conjugate base of the general formula (12) is used as the third compound, and a compound of the general formula (14) is used as the fourth compound.

Preferably, the solvent serving as the reaction field of the third compound and the fourth compound includes at least one selected from the group consisting of diethyl ether, tetrahydrofuran, dimethyl sulfoxide, acetonitrile, 2-methylpyrrolidinone, pyridine, picoline, lutidine, and dioxane.

In the preparation process B, it is preferable to add the third compound and the basic compound to the solvent and thereafter add the fourth compound to the solvent. If the fourth compound and the basic compound can coexist, it is also possible to add the fourth compound and the basic compound to the solvent and thereafter add the third compound to the solvent.

In the preparation processes A and B, the anionic leaving group (Lg, Lg′) is preferably one selected from the group consisting of chlorine, bromine, iodine, p-toluenesulfonate group (p-CH₃C₆H₄SO₃—), and trifluoromethanesulfonate group (CF₃SO₃—).

In the preparation processes A and B, the basic compound preferably includes at least one selected from the group consisting of sodium hydroxide, potassium hydroxide, sodium hydride, sodium metal, butyl lithium, lithium diisopropylamide, sodium carbonate, potassium carbonate, triethylamine, pyridine, picoline, lutidine, and sodium amide.

The invention can provide processes for preparing fluorine-containing alkoxyalkanes with high yields in which the production of by-products is suppressed. Also, the preparation processes of the invention allow easy purification of the fluorine-containing alkoxyalkanes. The preparation processes of the invention are therefore highly versatile.

While the novel features of the invention are set forth particularly in the appended claims, the invention, both as to organization and content, will be better understood and appreciated, along with other objects and features thereof, from the following detailed description taken in conjunction with the drawings.

### DETAILED DESCRIPTION OF THE INVENTION

The process for preparing a fluorine-containing alkoxyalkane of the invention (preparation process A) includes the step of reacting a first compound with a second compound in a basic compound or a solvent containing a basic compound. This process can produce, with a high yield, a fluorine-containing alkoxyalkane represented by the general formula (1):

R¹—O—R²—O—R³ (1)

where each of R¹ and R³ is a C₁ to C₆ alkyl group in which part of the hydrogen atoms may be replaced with one or more fluorine atoms, R² is a C₂ to C₄ alkylene group in which part of the hydrogen atoms may be replaced with one or more fluorine atoms, and at least one of R¹, R² and R³ contains one or more fluorine atoms.

The first compound is an alcohol with the highest acidity selected from the group consisting of the compounds represented by the general formulas (2) to (5):

R¹—OH (2)

R³—O—R²—OH (3)

R¹—O—R²—OH (4)

R³—OH (5)

The second compound is one selected from the group consisting of the compounds represented by the general formulas (6) to (9):

Lg-R²—O—R³ (6)

Lg-R¹ (7)

Lg-R³ (8)

Lg-R²—O—R¹ (9)

where Lg represents an anionic leaving group. The second compound reacts with the first compound to yield the fluorine-containing alkoxyalkane represented by the general formula (1).

According to the preparation process of the invention, nucleophilic substitution attack on the fluorine-containing alkoxyalkane by the conjugate base (alkoxide) of the first compound is seemingly suppressed. As a result, since the production of by-products is suppressed, the yield of the fluorine-containing alkoxyalkane can be improved and, in addition, the purification of the fluorine-containing alkoxyalkane becomes easy.

Specific examples of R¹ and R³ include a 2,2,2-trifluoroethyl group.

Specific examples of R² include an ethylene group.

Preferable examples of the anionic leaving group (Lg) include halogen atoms other than fluorine, such as chlorine, bromine, and iodine, a p-toluenesulfonate group (p-CH₃C₆H₄SO₃—), and a trifluoromethanesulfonate group (CF₃SO₃—).

Specific examples of the first compound include 2,2,2-trifluoroethanol.

Specific examples of the second compound include 2-methoxyethyl p-toluenesulfonate and 2-bromoethyl methyl ether.

In the preparation process A, the production of by-products is suppressed probably due to the following mechanism. An example in which the first compound is represented by the general formula (2) and the second compound is represented by the general formula (6) is explained below. In the solvent, the proton of the first compound is pulled out by the basic compound to form an alkoxide. The alkoxide makes a nucleophilic attack on the carbon bonded to the leaving group (Lg) in the second compound to cause elimination of the leaving group from the second compound. As a result, a fluorine-containing alkoxyalkane represented by the general formula (1) is produced. This reaction is represented by the formula [3].

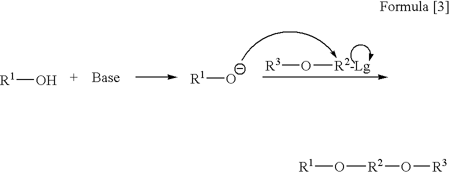

Meanwhile, between the fluorine-containing alkoxyalkane represented by the general formula (1) and the alkoxide (R¹—O—) produced from the compound represented by the general formula (2), four kinds of nucleophilic substitution reactions may occur. That is, four kinds of alkoxides may be eliminated from the fluorine-containing alkoxyalkane represented by the general formula (1). These side reactions are represented by the formula [4].

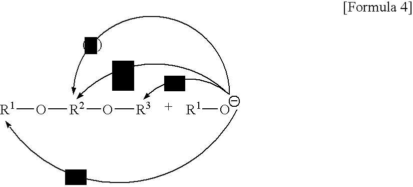

In the reaction (A), R¹—O—R²—O— is eliminated; in the reaction (B), R³—O— is eliminated; in the reaction (C), R¹—O— is eliminated; and in the reaction (D), R³—O—R²—O— is eliminated. However, in fact, of the conjugate acids of these alkoxides, R¹—O—R³—OH, R³—OH, R¹—OH, and R³—O—R²—OH, the higher acidity the hydroxyl group has, the more likely an alkoxide is eliminated therefrom. That is, when the hydroxyl group of R¹—OH has the highest acidity, even if the fluorine-containing alkoxyalkane underwent a nucleophilic attack by R¹—O—, R¹—O— is most likely to be eliminated. Hence, there is seemingly no change in the fluorine-containing alkoxyalkane. As a result, since the production of by-products can be suppressed, the purification of the fluorine-containing alkoxyalkane becomes easy.

The method for measuring the acidity of the hydroxyl group of an alcohol is not particularly limited; the acidity can be duly determined by those with common knowledge of organic chemistry or those skilled in the art. For example, the acidity can be directly obtained or indirectly estimated from the methods described in Non-Patent Documents 4 and 5, the tables contained therein, and the references cited therein.

In the second compound, the anionic leaving group (Lg) is not particularly limited. For example, those commonly used in organic synthesis are widely applicable. Among them, halogen atoms such as chlorine, bromine, and iodine, a p-toluenesulfonate group (p-CH₃C₆H₄SO₃—), and a trifluoromethanesulfonate group (CF₃SO₃—) are preferable due to their high reactivity.

The solvent serving as the reaction field of the first compound and the second compound is not particularly limited. For example, any one of the starting materials (i.e., any one of the first compound, the second compound, and the basic compound) may be excessively used so that it serves as the solvent. It should be noted, however, that the solvent is preferably stable with respect to the basic compound and the first compound. Specific examples include diethyl ether, tetrahydrofuran, dimethyl sulfoxide, acetonitrile, 2-methylpyrrolidinone, pyridine, picoline, lutidine, and dioxane. Also, in the case of previously preparing an alkoxide which is the conjugate base of the first compound, the solvent is preferably a basic compound such as pyridine, picoline, or lutidine, or the first compound itself. Since the alkoxide is highly soluble in such a solvent, uniform reaction can be carried out, so that the yield of the fluorine-containing alkoxyalkane is further improved.

In terms of, for example, facilitating the purification of the fluorine-containing alkoxyalkane, the boiling point of the solvent is preferably different from that of the desired fluorine-containing alkoxyalkane represented by the general formula (1) by 20 or more. For example, the boiling point of the solvent is more desirably higher than that of the fluorine-containing alkoxyalkane by 20 or more.

The basic compound is not particularly limited. Examples include alkali metals in the form of simple substance and compound, amines, and nitrogen-containing heteroaromatic compounds. Examples of alkali metals in the form of simple substance and compound include sodium hydroxide, potassium hydroxide, sodium hydride, sodium metal, butyl lithium, lithium diisopropylamide, sodium carbonate, and potassium carbonate. Examples of amines include triethylamine and sodium amide. Examples of nitrogen-containing heteroaromatic compounds include pyridine, picoline, and lutidine.

Among them, amines such as lithium diisopropylamide, triethylamine, pyridine, picoline, lutidine, and sodium amide, sodium hydride, sodium metal, butyl lithium, and the like do not produce water when they react. They are thus preferable in that the purification of the fluorine-containing alkoxyalkane becomes easier.

However, in the case of using an oxidative solvent (e.g., dimethyl sulfoxide), the basic compound is preferably at least one of sodium hydroxide, potassium hydroxide, and sodium hydride. When amines such as lithium diisopropylamide, triethylamine, pyridine, picoline, and lutidine are used, these basic compounds may be oxidized to form corresponding amine oxides.

The amount of the basic compound added is, for example, preferably 1 to 5 moles, and more preferably 1 to 2 moles per mole of the first compound. The second compound is, for example, preferably 1 to 5 moles, and more preferably 1 to 2 moles per mole of the first compound. The temperature of the solvent at which the first compound is reacted with the second compound is not particularly limited, but it is preferably −85 to 80° C.

In this preparation process, it is preferable to add the second compound to the solvent containing the first compound and the basic compound. Generally, an alkoxide dissolves in an alcohol that is the conjugate acid thereof, pyridine, and pyridine derivatives. Thus, a solvent containing an alkoxide can be prepared in advance by using the basic compound and the first compound. Thereafter, the second compound is added to the solvent containing the alkoxide. In this way, a fluorine-containing alkoxyalkane can be prepared efficiently.

For example, the basic compound is mixed with the solvent to form a suspension. The suspension is mixed with the first compound to form a mixed solution. At this time, the mixed solution is thought to contain an alkoxide which is the conjugate base of the first compound. The second compound is added to the mixed solution. This causes a nucleophilic substitution reaction between the alkoxide which is the conjugate base of the first compound and the second compound to yield a fluorine-containing alkoxyalkane. Thereafter, the fluorine-containing alkoxyalkane can be purified, for example, by fractional distillation under a reduced pressure of, for example, 20 kPa or less.

When the second compound and the basic compound can coexist in the solvent, it is also possible to add the first compound to a solvent containing the second compound and the basic compound. Examples of such combinations of the second compound and the basic compound include a combination of 2-methoxyethyl p-toluenesulfonate and sodium hydride and a combination of 2,2,2-trifluoroethyl p-toluenesulfonate and sodium hydroxide. By this, an alkoxide can be promptly produced from the first compound, and the produced alkoxide can be promptly reacted with the second compound. This method is particularly effective when the alkoxide produced from the first compound has a low solubility in the solvent.

For example, the basic compound is mixed with the solvent to form a suspension. The suspension is mixed with the second compound to form a mixed solution. The mixed solution is mixed with the first compound. By this, an alkoxide which is the conjugate base of the first compound is produced in the mixed solution, and a nucleophilic substitution reaction occurs between the alkoxide and the second compound to yield a fluorine-containing alkoxyalkane.

Another process (preparation process B) of the invention for preparing a fluorine-containing alkoxyalkane includes the step of reacting a third compound with a fourth compound in a basic compound or a solvent containing a basic compound. The process can produce, with a high yield, a fluorine-containing alkoxyalkane represented by the general formula (10):

R⁵—O—R⁴—O—R⁵ (10)

where R⁴ is a C₂ to C₄ alkylene group in which part of the hydrogen atoms may be replaced with one or more fluorine atoms, R⁵ is a C₁ to C₆ alkyl group in which part of the hydrogen atoms may be replaced with one or more fluorine atoms, and at least one of R⁴ and R⁵ includes one or more fluorine atoms.

It should be noted that the third compound is an alkoxide which is the conjugate base of an alcohol with a higher acidity selected from the group consisting of the compounds represented by the general formulas (11) and (12):

HO—R⁴—OH (11)

R⁵—OH (12)

The fourth compound is one selected from the group consisting of the compounds represented by the general formulas (13) and (14):

Lg-R⁵ (13)

Lg-R⁴-Lg′ (14)

where each of Lg and Lg′ represents an anionic leaving group. The fourth compound reacts with the third compound to yield a fluorine-containing alkoxyalkane represented by the general formula (10).

According to the preparation process B of the invention, the nucleophilic substitution attack on the fluorine-containing alkoxyalkane by the third compound is seemingly suppressed. Since the production of by-products is suppressed, the yield of the fluorine-containing alkoxyalkane can be improved and, in addition, the purification of the fluorine-containing alkoxyalkane becomes easy.

Specific examples of R⁵ include a 2,2,2-trifluoroethyl group.

Specific examples of R⁴ include an ethylene group.

Preferable examples of the anionic leaving group (Lg, Lg′) include halogen atoms other than fluorine, such as chlorine, bromine, and iodine, a p-toluenesulfonate group (p-CH₃C₆H₄SO₃—), and a trifluoromethanesulfonate group (CF₃SO₃—).

Specific examples of the third compound include tetrafluoro-1,2-diethoxide (—OCF₂CF₂O—).

Specific examples of the fourth compound include methyl p-toluenesulfonate and 2,2,2-trifluoroethyl p-toluenesulfonate.

In the preparation process B, the production of by-products is suppressed probably due to the following mechanism. An example in which the third compound is an alkoxide produced from an alcohol represented by the general formula (12) and the fourth compound is represented by the general formula (14) is explained below.

In the solvent, the alkoxide serving as the third compound makes a nucleophilic attack on the carbon bonded to the leaving group (Lg, Lg′) in the fourth compound to cause elimination of the leaving group from the fourth compound. As a result, a fluorine-containing alkoxyalkane represented by the general formula (10) is produced. This reaction is represented by the formula [5].

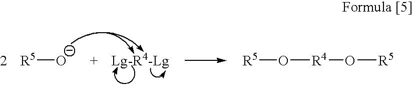

Meanwhile, between the fluorine-containing alkoxyalkane represented by the general formula (10) and the alkoxide (R⁵—O—) of the alcohol represented by the general formula (12), two kinds of nucleophilic substitution reactions may occur. That is, 2 kinds of alkoxides may be eliminated from the fluorine-containing alkoxyalkane represented by the general formula (10). Such a side reaction is represented by the formula [6].

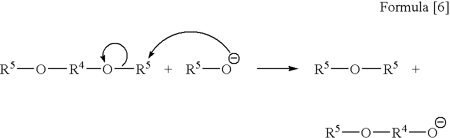

In the reaction of the formula [6], R⁵—O—R⁴—O— is eliminated. Meanwhile, in the reaction in which the alkoxide (R⁵—O—) attacks R⁴, R⁵—O— is eliminated. However, in fact, of the conjugate acids of these alkoxides, R⁵—O—R⁴—OH and R⁵—OH, the higher acidity the hydroxyl group has, the more likely an alkoxide is eliminated therefrom. That is, when the acidity of the hydroxyl group of R⁵—OH is higher than the acidity of the hydroxyl group of R⁵—O—R⁴—OH, even if the fluorine-containing alkoxyalkane underwent a nucleophilic attack by R⁵—O—, R⁵—O— is more likely to be eliminated. Hence, there is seemingly no change in the fluorine-containing alkoxyalkane. As a result, since the production of by-products can be suppressed, the purification of the fluorine-containing alkoxyalkane becomes easy.

In the fourth compound, the anionic leaving group (Lg, Lg′) is not particularly limited. For example, those commonly used in organic synthesis are widely applicable. Among them, halogen elements such as chlorine, bromine, and iodine, a p-toluenesulfonate group (p-CH₃C₆H₄SO₃—), and a trifluoromethanesulfonate group (CF₃SO₃—) are preferable due to their high reactivity.

The solvent serving as the reaction field of the third compound and the fourth compound is not particularly limited. For example, any one of the starting materials (i.e., any one of the third compound, the fourth compound, and the basic compound) may be excessively used so that it serves as the solvent. It should be noted, however, that the solvent is preferably stable with respect to the basic compound and the third compound. Specifically, diethyl ether, tetrahydrofuran, dimethyl sulfoxide, acetonitrile, 2-methylpyrrolidinone, pyridine, picoline, lutidine, dioxane, etc. can be used.

Also, in the case of previously preparing an alkoxide serving as the third compound from an alcohol represented by the general formula (12) or (13), the solvent is preferably a basic compound such as pyridine, picoline, or lutidine, or the third compound itself. Since the alkoxide is highly soluble in such a solvent, uniform reaction can be carried out, so that the yield of the fluorine-containing alkoxyalkane is further improved.

In terms of facilitating the purification of the fluorine-containing alkoxyalkane, the boiling point of the solvent is preferably different from that of the fluorine-containing alkoxyalkane represented by the general formula (10) by 20° C. or more. For example, the boiling point of the solvent is more desirably higher than that of the fluorine-containing alkoxyalkane by 20° C. or more.

The basic compound is not particularly limited. Examples include alkali metals in the form of simple substance and compound, amines, and nitrogen-containing heteroaromatic compounds. Examples of alkali metals in the form of simple substance and compound include sodium hydroxide, potassium hydroxide, sodium hydride, sodium metal, butyl lithium, lithium diisopropylamide, sodium carbonate, and potassium carbonate. Examples of amines include triethylamine and sodium amide. Examples of nitrogen-containing heteroaromatic compounds include pyridine, picoline, and lutidine.

Among them, amines such as lithium diisopropylamide, triethylamine, pyridine, picoline, lutidine, and sodium amide, sodium hydride, sodium metal, butyl lithium, and the like do not produce water when they react. They are thus preferable in that the purification of the fluorine-containing alkoxyalkane becomes easier.

However, in the case of using an oxidative solvent (e.g., dimethyl sulfoxide), the basic compound is preferably at least one of sodium hydroxide, potassium hydroxide, and sodium hydride.

The amount of the basic compound added is, for example, preferably 1 to 8 moles, and more preferably 1 to 3 moles per mole of the third compound. The fourth compound is, for example, preferably 0.2 to 10 moles, and more preferably 0.3 to 3 moles per mole of the third compound. The temperature of the solvent at which the third compound is reacted with the fourth compound is not particularly limited, but it is preferably −85 to 80° C.

In this preparation process, it is preferable to add the fourth compound to the solvent containing the third compound and the basic compound. Generally, an alkoxide dissolves in an alcohol that is the conjugate acid thereof, pyridine, and pyridine derivatives. Thus, a solvent containing an alkoxide as the third compound can be prepared in advance by using the basic compound and an alcohol represented by the general formula (11) or (12). Thereafter, the fourth compound is added to the solvent containing the alkoxide to yield a fluorine-containing alkoxyalkane. For example, the basic compound is mixed with the solvent to form a suspension. The suspension is mixed with an alcohol represented by the general formula (11) or (12) to form a mixed solution. At this time, the mixed solution is thought to contain an alkoxide as the third compound. The fourth compound is added to the mixed solution. This causes a nucleophilic substitution reaction between the alkoxide serving as the third compound and the fourth compound to yield a fluorine-containing alkoxyalkane. Thereafter, the fluorine-containing alkoxyalkane can be purified, for example, by fractional distillation under a reduced pressure of, for example, 20 kPa or less.

When the fourth compound and the basic compound can coexist in the solvent, it is also possible to add the third compound to a solvent containing the fourth compound and the basic compound. Examples of such combinations of the fourth compound and the basic compound include a combination of methyl p-toluenesulfonate and sodium hydride or sodium hydroxide and a combination of 2,2,2-trifluoroethyl p-toluenesulfonate and sodium hydride or sodium hydroxide. By this, an alkoxide can be promptly produced from an alcohol represented by the general formula (11) or (12), and the produced alkoxide can be promptly reacted with the fourth compound. This method is particularly effective when the alkoxide produced from an alcohol represented by the general formula (11) or (12) has a low solubility in the solvent.

For example, the basic compound is mixed with the solvent to form a suspension. The suspension is mixed with the fourth compound to form a mixed solution. The mixed solution is mixed with an alcohol represented by the general formula (11) or (12). By this, an alkoxide serving as the third compound is produced in the mixed solution, and a nucleophilic substitution reaction occurs between the alkoxide and the fourth compound to yield a fluorine-containing alkoxyalkane.

The invention is hereinafter described in derail by way of Examples and Comparative Examples.

### EXAMPLE 1

As a fluorine-containing alkoxyalkane represented by the general formula (1), 2,2,2-trifluoroethoxymethoxyethane (corresponding to the general formula (1) where R¹=CF₃CH₂—, R²=—CH₂CH₂—, and R³═CH₃—) was synthesized.

30 g of sodium hydride (purity>55%) available from Kanto Chemical Co., Inc. was put as a basic compound into a 500-mL three-necked flask equipped with a stirrer. The flask was sealed with a Dimroth condenser fitted with a dropping funnel and a three-way cock, and the gas inside the flask was replaced with argon.

A suspension was prepared by adding 250 mL of dehydrated dimethyl sulfoxide of Aldrich as a solvent into the flask. Using a syringe, 132 mL of 2-methoxyethyl p-toluenesulfonate (corresponding to the general formula (6) where R²=—CH₂CH₂—, R³═CH₃—, and Lg=p-toluenesulfonate group) available from Tokyo Chemical Industry Co., Ltd. was added as a second compound to the suspension to obtain a mixed solution. While the reaction system was kept near room temperature using a water bath, 50 mL of 2,2,2-trifluoroethanol (corresponding to the general formula (2) where R¹=CF₃CH₂—) was dropped as a first compound into the mixed solution using the dropping funnel to obtain a homogeneous solution. The homogeneous solution was stirred for 2 hours, and a low boiling-point component was collected under a reduced pressure by using a liquid nitrogen trap. In this way, 2,2,2-trifluoroethoxymethoxyethane was obtained.

The acidity (pKa) of the hydroxyl group of 2,2,2-trifluoroethanol in water is 12.4. Examples of alcohols which are the conjugate acids of alkoxides that may be eliminated from 2,2,2-trifluoroethoxymethoxyethane include 2-(2,2,2-trifluoroethoxy)ethanol, 2-methoxyethanol, and methanol. In these alcohols, the pKa of the hydroxyl group is 2-methoxyethanol:14.8 and methanol:15.5. 2-(2,2,2-trifluoroethoxy)ethanol, in which the methyl group of 2-methoxyethanol is replaced with a 2,2,2-trifluoroethyl group, is thought to exhibit a pKa value almost equivalent to that of 2-methoxyethanol, since the electron-withdrawing trifluoroethyl group is far away from the hydroxyl group. Hence, the acidity of the hydroxyl group of 2,2,2-trifluoroethanol was the highest.

### EXAMPLE 2

Dehydrated diethyl ether available from Kanto Chemical Co., Inc. was used as the solvent. Also, 60 mL of 2-bromoethyl methyl ether (corresponding to the general formula (6) where R²=—CH₂CH₂—, R³═CH₃—, and Lg=Br) available from Tokyo Chemical Industry Co., Ltd. was used as the second compound. Except for these, in the same manner as in Example 1, 2,2,2-trifluoroethoxymethoxyethane was prepared.

### EXAMPLE 3

30 g of sodium hydride (purity>55%) available from Kanto Chemical Co., Inc. was put as a basic compound into a 500-mL three-necked flask equipped with a stirrer. The flask was sealed with a Dimroth condenser fitted with a dropping funnel and a three-way cock, and the gas inside the flask was replaced with argon.

A suspension was prepared by adding 250 mL of β-picoline available from Kanto Chemical Co., Inc. into the flask. While the reaction system was kept near room temperature using a water bath, 50 mL of 2,2,2-trifluoroethanol (corresponding to the general formula (2) where R¹=CF₃CH₂—) was dropped as a first compound into the suspension using the dropping funnel to obtain a mixed solution. The dropping funnel was cleaned with 20 mL of pyridine, and then 132 mL of 2-methoxyethyl p-toluenesulfonate (corresponding to the general formula (6) where R²=—CH₂CH₂—, R³═CH₃—, and Lg=p-toluenesulfonate group) available from Tokyo Chemical Industry Co., Ltd. was further added as a second compound to the mixed solution using the dropping funnel. The resultant mixed solution containing the second compound was stirred for 2 hours, and a component with a boiling-point of approximately 70° C. was collected under a reduced pressure (20 kPa) by fractional distillation. In this way, 2,2,2-trifluoroethoxymethoxyethane was prepared.

### COMPARATIVE EXAMPLE 1

153 g of p-toluenesulfonyl chloride available from Kanto Chemical Co., Inc. was put into a 1000-mL three-necked flask equipped with a stirrer. The flask was sealed with a Dimroth condenser fitted with a dropping funnel and a three-way cock, and the gas inside the flask was replaced with argon.

Using a syringe, 330 mL of dehydrated pyridine available from Kanto Chemical Co., Inc. was added into the flask. While the reaction system was kept around room temperature using a water bath, 57.6 mL of 2,2,2-trifluoroethanol was dropped using the dropping funnel, to obtain a mixed solution. The mixed solution was stirred for 2 hours, and then, while the mixed solution was cooled with ice, approximately 200 mL of 5N hydrochloric acid was added thereto. The 5N hydrochloric acid was prepared by diluting concentrated hydrochloric acid available from Kanto Chemical Co., Inc. Approximately 200 mL of diethyl ether available from Kanto Chemical Co., Inc. was added to extract an organic layer, which was washed with water and then cleaned with 200 mL of a saturated solution of sodium hydrogen carbonate available from Kanto Chemical Co., Inc. After the cleaning, the organic layer was dried using anhydrous magnesium sulfate available from Kanto Chemical Co., Inc., and the diethyl ether was removed by using an evaporator. The main component of the resulting product was 2,2,2-trifluoroethyl p-toluenesulfonate. The yield of the 2,2,2-trifluoroethyl p-toluenesulfonate was approximately 95%.

27.6 g of sodium hydroxide available from Kanto Chemical Co., Inc. was put into a 500-mL three-necked flask equipped with a stirrer. The flask was sealed with a Dimroth condenser fitted with a dropping funnel and a three-way cock, and the gas inside the flask was replaced with argon.

250 mL of dehydrated dimethyl sulfoxide of Aldrich was added into the flask, and 132 mL of 2-methoxyethanol available from Kanto Chemical Co., Inc. was further added using a syringe.

Meanwhile, 50 mL of the product prepared in the above manner was dissolved in 100 mL of DMSO to prepare a solution.

While the reaction system was kept around room temperature using a water bath, the solution of the product prepared in the above manner was dropped into the flask using the dropping funnel to obtain a mixed solution. The mixed solution was stirred for 2 hours, and a low boiling-point component was collected under a reduced pressure using a liquid nitrogen trap. In this way, 2,2,2-trifluoroethoxymethoxyethane was prepared.

### COMPARATIVE EXAMPLE 2

30 g of Sodium hydride (purity>55%) available from Kanto Chemical Co., Inc. was put into a 500-mL three-necked flask equipped with a stirrer. The flask was sealed with a Dimroth condenser fitted with a dropping funnel and a three-way cock, and the gas inside the flask was replaced with argon.

250 mL of dehydrated pyridine available from Kanto Chemical Co., Inc. was added into the flask, and 132 mL of 2-methoxyethyl p-toluenesulfonate available from Tokyo Chemical Industry Co., Ltd. was further added using a syringe. While the reaction system was kept around room temperature using a water bath, 67 mL of 1-iodo-2,2,2-trifluoroethane of Fluorochem was dropped into the flask using the dropping funnel. After the dropping, the stirring was further continued for 2 hours, and a low boiling-point component was collected under a reduced pressure, using a liquid nitrogen trap. In this way, 2,2,2-trifluoroethoxymethoxyethane was prepared.

The products obtained in Examples 1 to 3 and Comparative Examples 1 to 2 were analyzed by ¹H-NMR. From the weights of the products and the molar ratios obtained by NMR, the yields in Examples 1 to 3 and Comparative Examples 1 to 2 were determined. The results are shown in Table 1.

TABLE 1

| | Yield of | |
|---|---|---|
| | desired | |
| | product | By-product |
| Example 1 | 85% | (Dimethyl sulfoxide) |
| Example 2 | 85% | (Diethyl ether) |
| Example 3 | 80% | (β-picoline) |
| Comparative | 8% | Diglyme |
| Example 1 | | Bis(2,2,2-trifluoroethyl)ether |
| | | Water |
| | | (Dimethyl sulfoxide) |
| Comparative | 5% | Diglyme |
| Example 2 | | Bis(2,2,2-trifluoroethyl)ether |
| | | (Dimethyl sulfoxide) |

In all of Examples 1 to 3, the yield of 2,2,2-trifluoroethoxymethoxyethane was more than 80%, which is a good value. Also, no peaks of by-products appeared in the ¹H-NMR analysis.

On the other hand, in both Comparative Examples 1 and 2, the yield of 2,2,2-trifluoroethoxymethoxyethane was less than 10%. Also, the ¹H-NMR analysis of the products of Comparative Examples 1 and 2 showed the production of diglyme and bis(2,2,2-trifluoroethyl)ether. The cause is probably as follows.

First, the desired product 2,2,2-trifluoroethoxymethoxyethane reacts with 2-methoxyethoxide to cause elimination of 2,2,2-trifluoroethoxide. Thereafter, as shown by the formula [7], 2,2,2-trifluoroethoxide makes a nucleophilic attack on 2,2,2-trifluoroethyl p-toluenesulfonate. As a result, it is thought that diglyme and bis(2,2,2-trifluoroethyl)ether were produced.

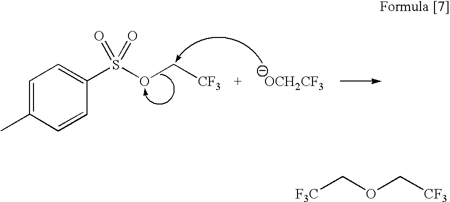

2,2,2-trifluoroethoxide has an electron withdrawing trifluoromethyl group. Thus, 2,2,2-trifluoroethoxide is more stable than 2-methoxyethoxide and is more likely to be eliminated. Probably for this reason, the reaction shown by the formula [7] proceeded predominantly. As described above, the yield is improved by selecting the most stable one from all the alkoxides that may be produced as the alkoxide serving as the nucleophilic reagent. In other words, it is effective to use an alcohol whose hydroxyl group has the highest acidity as the first compound.

As described above, it has been shown that the invention can suppress the production of by-products and provide 2,2,2-trifluoroethoxymethoxyethane with high yields. Since the production of by-products is suppressed, the purification of 2,2,2-trifluoroethoxymethoxyethane was easy.

### EXAMPLE 4

As a fluorine-containing alkoxyalkane represented by the general formula (10), bis(2,2,2-trifluoroethoxy)ethane (corresponding to the general formula (10) where R⁵═CF₃CH₂— and R⁴=—CH₂CH₂—) was synthesized.

First, a fourth compound was prepared in the following manner. 153 g of p-toluenesulfonyl chloride available from Kanto Chemical Co., Inc. was put into a 1000-mL three-necked flask equipped with a stirrer. The flask was sealed with a Dimroth condenser fitted with a dropping funnel and a three-way cock, and the gas inside the flask was replaced with argon.

Using a syringe, 330 mL of dehydrated pyridine available from Kanto Chemical Co., Inc. was added into the flask. While the reaction system was kept around room temperature using a water bath, 57 mL of 2-bromoethanol was dropped into the flask using the dropping funnel, to obtain a mixed solution. The mixed solution was stirred for 2 hours, and then, while the mixed solution was cooled with ice, approximately 200 mL of 5N hydrochloric acid was added thereto. The 5N hydrochloric acid was prepared by diluting concentrated hydrochloric acid available from Kanto Chemical Co., Inc. Approximately 200 mL of diethyl ether available from Kanto Chemical Co., Inc. was added to extract an organic layer, which was washed with water and then cleaned with 200 mL of a saturated solution of sodium hydrogen carbonate available from Kanto Chemical Co., Inc. After the cleaning, the organic layer was dried using anhydrous magnesium sulfate available from Kanto Chemical Co., Inc., and the diethyl ether was removed by using an evaporator. In this way, 2-bromoethyl p-toluenesulfonate (corresponding to the general formula (14) where R⁴=—CH₂CH₂—, Lg=Br, and Lg′=p-toluenesulfonate group) used as the fourth compound was prepared. The yield of 2-bromoethyl p-toluenesulfonate was approximately 95%.

30 g of sodium hydride (purity>55%) available from Kanto Chemical Co., Inc. was put as a basic compound into a 500-mL three-necked flask equipped with a stirrer. The flask was sealed with a Dimroth condenser fitted with a dropping funnel and a three-way cock, and the gas inside the flask was replaced with argon.

A suspension was prepared by adding 250 mL of β-picoline available from Kanto Chemical Co., Inc. into the flask. While the reaction system was kept near room temperature using a water bath, 50 mL of 2,2,2-trifluoroethanol (corresponding to the general formula (12) where R⁵═CF₃CH₂—) was dropped into the suspension using the dropping funnel, to obtain a mixed solution. In the mixed solution, 2,2,2-trifluoroethoxide serving as the third compound was produced from 2,2,2-trifluoroethanol.

Meanwhile, 96.3 g of 2-bromoethyl p-toluenesulfonate was dissolved as the fourth compound in 150 mL of β-picoline. This solution was added to the mixed solution using the dropping funnel that had been cleaned with 20 mL of pyridine. The mixed solution containing the fourth compound was stirred for 2 hours, and a component with a boiling point of approximately 70° C. was collected by fractional distillation under a reduced pressure (20 kPa).

### COMPARATIVE EXAMPLE 3

30 g of sodium hydride (purity>55%) available from Kanto Chemical Co., Inc. and 175 g of 2,2,2-trifluoroethyl p-toluenesulfonate (corresponding to the general formula (13) where R⁵═CF₃CH₂— and Lg=p-toluenesulfonate group) serving as the fourth compound were put into a 500-mL three-necked flask equipped with a stirrer. The flask was sealed with a Dimroth condenser fitted with a dropping funnel and a three-way cock, and the gas inside the flask was replaced with argon.

A suspension was prepared by adding 250 mL of dehydrated dimethyl sulfoxide available from Kanto Chemical Co., Inc. into the flask. While the reaction system was kept around room temperature using a water bath, 19 mL of dehydrated ethylene glycol (pKa: 15.4 (in water)) of Aldrich was dropped into the flask using the dropping funnel, to obtain a mixed solution. The mixed solution was stirred for 2 hours, and a low boiling-point component was collected under a reduced pressure using a liquid nitrogen trap. However, bis(2,2,2-trifluoroethoxy)ethane was not obtained.

The products obtained in Example 4 and Comparative Example 3 were analyzed by ¹H-NMR. From the weights of the products and the molar ratios obtained from NMR, the yields in Example 4 and Comparative Example 3 were determined. The results are shown in Table 2.

TABLE 2

| | Yield of | |
|---|---|---|
| | desired | |
| | product | By-product |
| Example 4 | 75% | (β-picoline) |
| Comparative | 0% | Ethylene oxide |
| Example 3 | | bis(2,2,2-trifluoroethyl)ether |
| | | (Dimethyl sulfoxide) |

In Example 4, the yield of bis(2,2,2-trifluoroethoxy)ethane was 75%, which is a good value. Also, in the ¹H-NMR analysis, except for β-picoline used as the solvent, no peaks of by-products appeared.

On the other hand, in Comparative Example 3, the desired compound bis(2,2,2-trifluoroethoxy)ethane was not obtained at all. Also, the ¹H-NMR analysis showed the production of ethylene oxide and bis(2,2,2-trifluoroethyl)ether. The cause is probably as follows.

First, as shown by the formula [8], the dialkoxide produced from ethylene glycol makes a nucleophilic attack on 2,2,2-trifluoroethyl p-toluenesulfonate. As a result, it is thought that 2,2,2-trifluoroethylethoxide was produced as an intermediate, and that intramolecular cyclization of the intermediate occurred.

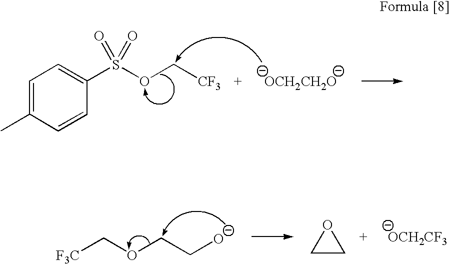

The fluorine substituent is closer to the anion moiety in 2,2,2-trifluoroethoxide than in the intermediate 2,2,2-trifluoroethylethoxide. Thus, 2,2,2-trifluoroethoxide is more stable than 2,2,2-trifluoroethylethoxide. Probably for this reason, intramolecular cyclization as shown by the formula [8] proceeds.

The thus produced 2,2,2-trifluoroethoxide makes a nucleophilic attack on 2,2,2-trifluoroethyl p-toluenesulfonate. As a result, it is believed that bis(2,2,2-trifluoroethyl)ether was produced.

As described above, it has been found that the use of the alkoxide derived from the alcohol whose hydroxyl group has the highest acidity among HOCH₂CH₂OH, CF₃CH₂OCH₂CH₂OH, and CF₃CH₂OH is effective for suppressing the production of by-products.

### EXAMPLE 5

As a fluorine-containing alkoxyalkane represented by the general formula (10), 1,2-dimethoxy-1,1,2,2-tetrafluoroethane (corresponding to the general formula (10) where R⁵=—CH₃ and R⁴=—CF₂CF₂—) was synthesized.

8.73 g of sodium hydride (purity>55%) available from Kanto Chemical Co., Inc. was put into a 200-mL three-necked flask equipped with a stirrer. The flask was sealed with a Dimroth condenser fitted with a dropping funnel and a three-way cock, and the gas inside the flask was replaced with argon.

A suspension was prepared by adding 80 mL of dehydrated dimethyl sulfoxide available from Kanto Chemical Co., Inc. into the flask. While the reaction system was kept around room temperature using a water bath, 8.1 mL of dehydrated methanol of Aldrich was dropped into the flask using the dropping funnel, to obtain a mixed solution.

6.7 mL of 1,2-diiodotetrafluoroethane (compound that produces a dialkoxide (third compound) corresponding to the general formula (11) where R⁴=—CF₂CF₂—) of Alfa Aesar was dropped into the mixed solution, which was then stirred for 2 hours. Subsequently, a solution prepared by dissolving 18.6 g of methyl p-toluenesulfonate (corresponding to the general formula (13) where R⁵=—CH₃ and Lg=p-toluenesulfonate group) available from Tokyo Chemical Industry Co., Ltd. in 10 mL of dimethyl sulfoxide was dropped into the mixed solution, which was then stirred for 2 hours. A low boiling-point component was collected under a reduced pressure using a liquid nitrogen trap.

### COMPARATIVE EXAMPLE 4

4.36 g of sodium hydride (purity>55%) available from Kanto Chemical Co., Inc. was put into a 200-mL three-necked flask equipped with a stirrer. The flask was sealed with a Dimroth condenser fitted with a dropping funnel and a three-way cock, and the gas inside the flask was replaced with argon.

A suspension was prepared by adding 80 mL of dehydrated dimethyl sulfoxide available from Kanto Chemical Co., Inc. into the flask. While the reaction system was kept around room temperature using a water bath, 4.0 mL of dehydrated methanol of Aldrich was dropped into the flask using the dropping funnel, to obtain a mixed solution.

6.7 mL of 1,2-diiodotetrafluoroethane of Alfa Aesar was dropped into the mixed solution, which was then stirred for 2 hours. A low boiling-point component was collected under a reduced pressure using a liquid nitrogen trap.

The products obtained in Example 5 and Comparative Example 4 were analyzed by ¹H-NMR. From the weights of the products and the molar ratios obtained from NMR, the yields in Example 5 and Comparative Example 4 were determined. The results are shown in Table 3.

TABLE 3

| | Yield of | |
|---|---|---|
| | desired | |
| | product | By-product |
| Example 5 | 75% | Dimethyl ether |
| | | (Dimethyl sulfoxide) |
| Comparative | 5% | Dimethyl ether |
| Example 4 | | (Dimethyl sulfoxide) |

In Example 5, the yield of 1,2-dimethoxy-1,1,2,2-tetrafluoroethane was 75%, which is a good value. On the other hand, in Comparative Example 4, the yield of 1,2-dimethoxy-1,1,2,2-tetrafluoroethane was 5%. In either case, the ¹H-NMR analysis confirmed the presence of dimethyl ether as a by-product.

In Comparative Example 4, since two equivalents of sodium hydride and methanol were used relative to 1,2-diiodotetrafluoroethane, the reaction represented by the formula [9] was expected to occur.

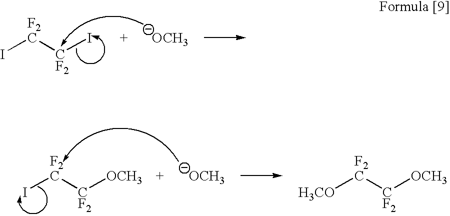

However, in fact, as shown by the formula [10], it is thought that the produced 1,2-dimethoxy-1,1,2,2-tetrafluoroethane underwent a nucleophilic attack by methoxide, thereby resulting in the production of dimethyl ether and 2-methoxy-1,1,2,2-tetrafluoroethoxide. This is probably because the CH₃OCF₂CF₂O— anion having an electron withdrawing CF₂CF₂ group is more stable than the methoxide anion and is more likely to be eliminated. That is, in a comparison of the acidities of the hydroxyl groups of the conjugate acids CH₃OH and CH₃OCF₂CF₂OH, CH₃OCF₂CF₂OH has a higher acidity.

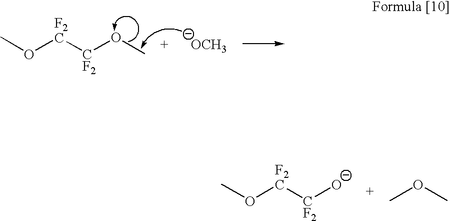

On the other hand, in Example 5 of the invention, 4 equivalents of sodium hydride and methanol were used relative to 1,2-diiodotetrafluoroethane. Thus, the reaction represented by the formula [10] proceeds further to form an activated species of —OCF₂CF₂O— as shown by the formula [11]. This species makes a nucleophilic attack on methyl p-toluenesulfonate, and this is probably the reason why 1,2-dimethoxy-1,1,2,2-tetrafluoroethane could be synthesized with a good yield.

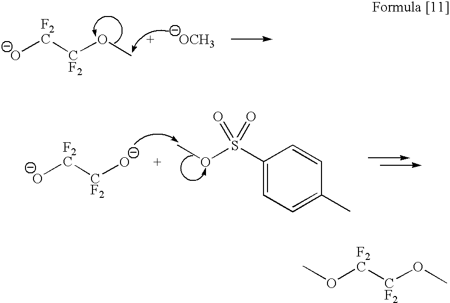

As described above, it has been found that the production of by-products can be suppressed by reacting an alkoxide which is the conjugate base of an alcohol whose hydroxyl group has a higher acidity selected from HO—R⁴—OH and R⁵—OH with a corresponding compound represented by Lg-R⁵ or Lg-R⁴-Lg′.

As described above, according to the invention, fluorine-containing alkoxyalkanes preferable as non-aqueous electrolytes included in non-aqueous electrolyte secondary batteries can be prepared with high yields. The invention can therefore contribute to an improvement in the performance of non-aqueous electrolyte secondary batteries and a cost reduction.

Although the invention has been described in terms of the presently preferred embodiments, it is to be understood that such disclosure is not to be interpreted as limiting. Various alterations and modifications will no doubt become apparent to those skilled in the art to which the invention pertains, after having read the above disclosure. Accordingly, it is intended that the appended claims be interpreted as covering all alterations and modifications as fall within the true spirit and scope of the invention.

1. A process for preparing a fluorine-containing alkoxyalkane represented by the general formula (1):
R¹—O—R²—O—R³ (1)
where each of R¹ and R³ is a C₁ to C₆ alkyl group in which part of the hydrogen atoms may be replaced with one or more fluorine atoms, R² is a C₂ to C₄ alkylene group in which part of the hydrogen atoms may be replaced with one or more fluorine atoms, and at least one of R¹, R² and R³ contains one or more fluorine atoms,
said process comprising the step of reacting a first compound with a second compound in a basic compound or a solvent containing a basic compound, said second compound being reactive with said first compound,
wherein said first compound is an alcohol with the highest acidity selected from the group consisting of the compounds represented by the general formulas (2) to (5):
R¹—OH (2)
R³—O—R²—OH (3)
R¹—O—R²—OH (4)
R³—OH (5)
said second compound is one selected from the group consisting of the compounds represented by the general formulas (6) to (9):
Lg-R²—O—R³ (6)
Lg-R¹ (7)
Lg-R³ (8)
Lg-R²—O—R¹ (9)
where Lg represents an anionic leaving group, and
said second compound is a compound that reacts with said first compound to yield said fluorine-containing alkoxyalkane.

2. The process for preparing a fluorine-containing alkoxyalkane in accordance with claim 1, wherein the combination of said first compound and said second compound is a combination of compounds of the general formula (2) and the general formula (6), a combination of compounds of the general formula (3) and the general formula (7), a combination of compounds of the general formula (4) and the general formula (8), or a combination of compounds of the general formula (5) and the general formula (9).

3. The process for preparing a fluorine-containing alkoxyalkane in accordance with claim 1, wherein said leaving group is one selected from the group consisting of chlorine, bromine, iodine, p-toluenesulfonate group (p-CH₃C₆H₄SO₃—), and trifluoromethanesulfonate group (CF₃SO₃—).

4. The process for preparing a fluorine-containing alkoxyalkane in accordance with claim 1, wherein said solvent includes at least one selected from the group consisting of diethyl ether, tetrahydrofuran, dimethyl sulfoxide, 2-methylpyrrolidinone, pyridine, picoline, lutidine, and dioxane.

5. The process for preparing a fluorine-containing alkoxyalkane in accordance with claim 1, wherein said basic compound includes at least one selected from the group consisting of sodium hydroxide, potassium hydroxide, sodium hydride, sodium metal, butyl lithium, lithium diisopropylamide, sodium carbonate, potassium carbonate, triethylamine, pyridine, picoline, and lutidine.

6. The process for preparing a fluorine-containing alkoxyalkane in accordance with claim 1, wherein after said first compound and said basic compound are added to said solvent, said second compound is added to said solvent.

7. The process for preparing a fluorine-containing alkoxyalkane in accordance with claim 1, wherein after said second compound and said basic compound are added to said solvent, said first compound is added to said solvent.

8. A process for preparing a fluorine-containing alkoxyalkane represented by the general formula (10):
R⁵—O—R⁴—O—R⁵ (10)
where R⁴ is a C₂ to C₄ alkylene group in which part of the hydrogen atoms may be replaced with one or more fluorine atoms, R⁵ is a C₁ to C₆ alkyl group in which part of the hydrogen atoms may be replaced with one or more fluorine atoms, and at least one of R⁴ and R⁵ includes one or more fluorine atoms,
said process comprising the step of reacting a third compound with a fourth compound in a basic compound or a solvent containing a basic compound, said fourth compound being reactive with said third compound,
wherein said third compound is an alkoxide which is the conjugate base of an alcohol with a higher acidity selected from the group consisting of the compounds represented by the general formulas (11) and (12):
HO—R⁴—OH (11)
R⁵—OH (12),
said fourth compound is one selected from the group consisting of the compounds represented by the general formulas (13) and (14):
Lg-R⁵ (13)
Lg-R⁴-Lg′ (14)
where each of Lg and Lg′ is an anionic leaving group, and
said fourth compound is a compound that reacts with said third compound to yield said fluorine-containing alkoxyalkane.

9. The process for preparing a fluorine-containing alkoxyalkane in accordance with claim 8, wherein the combination of said third compound and said fourth compound is a combination of an alkoxide which is the conjugate base of the general formula (11) and a compound of the general formula (13), or a combination of an alkoxide which is the conjugate base of the general formula (12) and a compound of the general formula (14).

10. The process for preparing a fluorine-containing alkoxyalkane in accordance with claim 8, wherein said leaving group is one selected from the group consisting of chlorine, bromine, iodine, p-toluenesulfonate group (p-CH₃C₆H₄SO₃—), and trifluoromethanesulfonate group (CF₃SO₃—).

11. The process for preparing a fluorine-containing alkoxyalkane in accordance with claim 8, wherein said solvent includes at least one selected from the group consisting of diethyl ether, tetrahydrofuran, dimethyl sulfoxide, 2-methylpyrrolidinone, pyridine, picoline, lutidine, and dioxane.

12. The process for preparing a fluorine-containing alkoxyalkane in accordance with claim 8, wherein said basic compound includes at least one selected from the group consisting of sodium hydroxide, potassium hydroxide, sodium hydride, sodium metal, butyl lithium, lithium diisopropylamide, sodium carbonate, potassium carbonate, triethylamine, pyridine, picoline, lutidine, and sodium amide.

13. The process for preparing a fluorine-containing alkoxyalkane in accordance with claim 8, wherein after said third compound and said basic compound are added to said solvent, said fourth compound is added to said solvent.

14. The process for preparing a fluorine-containing alkoxyalkane in accordance with claim 8, wherein after said fourth compound and said basic compound are added to said solvent, said third compound is added to said solvent.
